(19) United States
(12) Patent Application Publication
(10) Pub. No.: US 2022/0343160 A1
(43) Pub. Date: Oct. 27, 2022

(54) TIME SERIES DATA PROCESSING DEVICE CONFIGURED TO PROCESS TIME SERIES DATA WITH IRREGULARITY

(71) Applicant: Electronics and Telecommunications Research Institute, Daejeon (KR)

(72) Inventors: Hwin Dol PARK, Daejeon (KR); Jae Hun CHOI, Daejeon (KR); Youngwoong HAN, Daejeon (KR)

(73) Assignee: Electronics and Telecommunications Research Institute, Daejeon (KR)

(21) Appl. No.: 17/725,172

(22) Filed: Apr. 20, 2022

(30) Foreign Application Priority Data
Apr. 22, 2021 (KR) 10-2021-0052485

(51) Int. Cl. G06N 3/08

(52) U.S. Cl. CPC G06N 3/08

(57) ABSTRACT

Disclosed is a time series data processing device which includes a pre-processor that performs pre-processing on time series data to generate pre-processing data, and a learner that creates or updates a feature model through machine learning for the pre-processing data. The learner includes a time series irregularity learning model that learns time series irregularity of the pre-processing data, and a feature irregularity learning model that learns feature irregularity of the pre-processing data.

### CROSS-REFERENCE TO RELATED APPLICATIONS

This application claims priority under 35 U.S.C. § 119 to Korean Patent Application No. 10-2021-0052485 filed on Apr. 22, 2021, in the Korean Intellectual Property Office, the disclosures of which are incorporated by reference herein in their entireties.

### BACKGROUND

Embodiments of the present disclosure described herein relate to a data processing device, and more particularly, relate to a time series data processing device configured to process time series data with irregularity.

With the development of various technologies including a medical technology, the standard of living of humans is improved, and the lifespan of humans is increasing. However, with the development of various technologies, changes in lifestyle and wrong eating habits cause various diseases. To lead a healthy life, there is a demand on predicting future health status in addition to treating current diseases. As such, there is being developed a method for predicting the health status at a future time by analyzing the development of changes in time series medical data over time.

As industrial technologies and information and communication technologies develop, a significant amount of information and data is being generated. Nowadays, there is emerging a technology for artificial intelligence that trains an electronic device (e.g., a computer) by using such a large amount of information and data such that various services are provided. In particular, to predict the future health status, a method for building a prediction model using various time series medical data is being developed. For example, time series medical data differs from data collected in other fields in that the time series medical data have irregular time periods and include missing values and complex and unspecified features. Accordingly, there is a demand on effectively processing and analyzing time series medical data for the purpose of predicting the future health status.

### SUMMARY

Embodiments of the present disclosure provide a time series data processing device configured to process time series data with irregularity by predicting various measurement period points in time with respect to time series data with time series irregularity and feature irregularity through measurement period division and change development modeling.

According to an embodiment, a time series data processing device includes a pre-processor that performs pre-processing on time series data to generate pre-processing data, and a learner that creates or updates a feature model through machine learning for the pre-processing data. The learner includes a time series irregularity learning model that learns time series irregularity of the pre-processing data, and a feature irregularity learning model that learns feature irregularity of the pre-processing data.

In an embodiment, the pre-processor includes a numerical data normalizing unit that normalizes the time series data to generate a plurality of feature data, a first missing value processing unit that replaces a missing value of first feature data of the plurality of feature data with a specific value, and a missing value mask generating unit that generates mask data based on a missing value of the plurality of feature data.

In an embodiment, the specific value is decided based on at least one of a value corresponding to next feature data associated with a feature corresponding to the missing value of the first feature data of the plurality of feature data, an average value, a median value, a central value, a maximum value, or a minimum value, a value based on a machine learning technique.

In an embodiment, the pre-processor further includes a measurement period calculating unit that calculates a period of the time series data, and a measurement period converting unit that converts the period calculated from the measurement period calculating unit into a minimum unit to output a measurement period, and the pre-processing data include the plurality of feature data, the measurement period, and the mask data.

In an embodiment, the time series irregularity learning model includes a time series sequence processing unit that embeds the plurality of feature data of the pre-processing data to output a plurality of embedding data, a measurement period processing unit that divides the measurement period into a plurality of sub periods, and a time series calculating unit that calculates a plurality of first prediction data respectively associated with the plurality of sub periods based on first embedding data of the plurality of embedding data.

In an embodiment, the time series calculating unit estimates a first slope based on a first sub period of the plurality of sub periods and the first embedding data, calculates one prediction data of the plurality of first prediction data based on the first slope, the first sub period, and the first embedding data, estimates a second slope based on a second sub period of the plurality of sub periods and the one prediction data, and calculates another prediction data of the plurality of first prediction data based on the second slope, the second sub period, and the one prediction data.

In an embodiment, the first slope and the second slope are estimated based on a neural network estimating a function of a slope of a distribution of the plurality of feature data.

In an embodiment, the feature irregularity learning model includes a missing value mask processing unit that generates masked prediction data based on last prediction data of the plurality of first prediction data and the mask data, and a missing value replacement applying unit that generates replacement data by replacing a missing value of feature data corresponding to the masked prediction data from among the plurality of feature data, based on the masked prediction data.

In an embodiment, the time series calculating unit calculates a plurality of second prediction data respectively associated with the plurality of sub periods based on the replacement data.

In an embodiment, the time series data processing device further includes a feature ground processing unit that performs a first neural network operation on the plurality of first prediction data and the plurality of second prediction data to decide a feature weight, and applies the feature weight to the plurality of first prediction data and the plurality of second prediction data to generate data to which the feature weight is applied, and the feature weight indicates a correlation between the plurality of feature data.

In an embodiment, the time series data processing device further includes a time series ground processing that performs a second neural network operation on the plurality of first prediction data and the plurality of second prediction data to decide a time series weight, and applies the time series weight to the plurality of first prediction data and the plurality of second prediction data to generate data to which the time series weight is applied, and the time series weight indicates a correlation associated with the period of the time series data.

According to an embodiment, a time series data processing device includes a pre-processor that performs pre-processing on time series data to generate pre-processing data, and a predictor that performs machine learning on the pre-processing data based on a feature model and to output a prediction result and prediction grounds. The predictor includes a time series irregularity predicting module that calculates a plurality of prediction data based on the feature model and a sub period smaller than a measurement period of the pre-processing data, a feature irregularity predicting module that replaces a missing value of the pre-processing data based on the plurality of prediction data, and a ground tracking predicting module that generates a feature weight and a time series weight based on the plurality of prediction data, to apply the feature weight and the time series weight to the plurality of prediction data, and to output data to which a weight is applied, and the prediction result includes at least one of the plurality of prediction data, and the prediction grounds include the data to which the weight is applied.

In an embodiment, the pre-processor includes a numerical data normalizing unit that normalizes the time series data to generate a plurality of feature data, a first missing value processing unit that replaces a missing value of first feature data of the plurality of feature data with a specific value, and a missing value mask generating unit that generates mask data based on a missing value of the plurality of feature data.

In an embodiment, the pre-processor further includes a measurement period calculating unit that calculates a period of the time series data, and a measurement period converting unit that converts the period calculated from the measurement period calculating unit into a minimum unit to output a measurement period, and the pre-processing data include the plurality of feature data, the measurement period, and the mask data.

In an embodiment, the time series irregularity predicting model includes a time series sequence processing unit that embeds the plurality of feature data of the pre-processing data to output a plurality of embedding data, a measurement period processing unit that divides the measurement period into a plurality of sub periods, and a time series calculating unit that calculates a plurality of first prediction data respectively associated with the plurality of sub periods based on first embedding data of the plurality of embedding data.

In an embodiment, the time series calculating unit estimates a first slope based on a first sub period of the plurality of sub periods and the first embedding data, calculates one prediction data of the plurality of first prediction data based on the first slope, the first sub period, and the first embedding data, estimates a second slope based on a second sub period of the plurality of sub periods and the one prediction data, and calculates another prediction data of the plurality of first prediction data based on the second slope, the second sub period, and the one prediction data.

In an embodiment, the feature irregularity predicting module includes a missing value mask processing unit that generates masked prediction data based on last prediction data of the plurality of first prediction data and the mask data, and a missing value replacement applying unit that generates replacement data by replacing a missing value of feature data corresponding to the masked prediction data from among the plurality of feature data, based on the masked prediction data.

In an embodiment, the time series calculating unit calculates a plurality of second prediction data respectively associated with the plurality of sub periods based on the replacement data.

### BRIEF DESCRIPTION OF THE FIGURES

The above and other objects and features of the present disclosure will become apparent by describing in detail embodiments thereof with reference to the accompanying drawings.

FIG. 1 is a block diagram of a time series data processing device according to an embodiment of the present disclosure.

FIG. 2 is a diagram for describing time series irregularity and feature irregularity of time series data described with reference to FIG. 1.

FIG. 3 is a block diagram illustrating a pre-processor of FIG. 1.

FIGS. 4 and 5 are diagrams for describing a pre-processing operation of a pre-processor of FIG. 3.

FIG. 6 is a block diagram illustrating a learner of FIG. 1.

FIG. 7 is a block diagram illustrating a time series irregularity learning module of FIG. 6.

FIG. 8 is a diagram for describing an operation of a measurement period processing unit of FIG. 7.

FIG. 9 is a diagram for describing an operation of a time series calculating unit of FIG. 7.

FIG. 10 is a block diagram illustrating a feature irregularity learning module of FIG. 6.

FIG. 11 is a diagram for describing operations of a time series irregularity learning module and a feature irregularity learning module of FIG. 6.

FIG. 12 is a block diagram illustrating a ground tracking learning module of FIG. 6.

FIG. 13 is a block diagram illustrating a predictor of FIG. 1.

FIG. 14 is a diagram illustrating a health status predicting system to which a time series data processing device of FIG. 1 is applied.

FIG. 15 is a block diagram of a time series data processing device of FIG. 1 or 14.

### DETAILED DESCRIPTION

Below, embodiments of the present disclosure will be described in detail and clearly to such an extent that one skilled in the art easily carries out the present disclosure.

Components described in the specification by using the terms “part”, “unit”, “module”, “engine”, etc. and function blocks illustrated in drawings may be implemented with software, hardware, or a combination thereof. For example, the software may be a machine code, firmware, an embedded code, and application software. For example, the hardware may include an electrical circuit, an electronic circuit, a processor, a computer, an integrated circuit, integrated circuit cores, a pressure sensor, an inertial sensor, a microelectromechanical system (MEMS), a passive element, or a combination thereof.

Also, unless differently defined, all terms used herein, which include technical terminologies or scientific terminologies, have the same meaning as that understood by a person skilled in the art to which the inventive concept belongs. Terms defined in a generally used dictionary are to be interpreted to have meanings equal to the contextual meanings in a relevant technical field, and are not interpreted to have ideal or excessively formal meanings unless clearly defined in the specification.

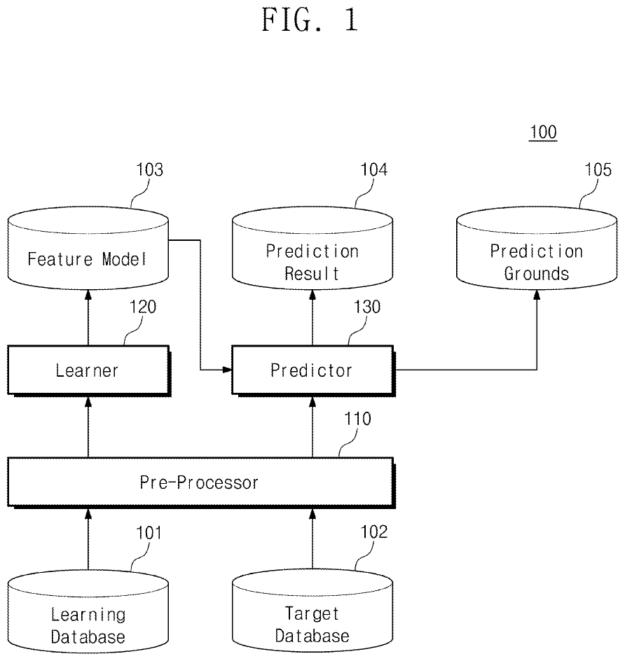

FIG. 1 is a block diagram of a time series data processing device according to an embodiment of the present disclosure. A time series data processing device 100 of FIG. 1 may be understood as an example configuration for pre-processing time series data and training a prediction model based on a result of analyzing the pre-processed time series data or generating a prediction result and prediction grounds. In an embodiment, in consideration of the time series irregularity of time series data with irregular time periods, the time series data processing device 100 may predict a future time and may provide prediction grounds associated with a prediction result. Alternatively, the time series data processing device 100 may predict various prediction points in time through the modeling of the development of time series changes, in a data environment in which measurement period information is limited.

Referring to FIG. 1, the time series data processing device 100 may include a pre-processor 110, a learner 120, and a predictor 130. The pre-processor 110, the learner 120, and the predictor 130 may be implemented with hardware or may be implemented with firmware, software, or a combination thereof. For example, the software (or firmware) may be loaded onto a memory (not illustrated) included in the time series data processing device 100 and may be executed by a processor (not illustrated). For example, the pre-processor 110, the learner 120, and the predictor 130 may be implemented with a field programmable gate array (FPGA), an application specific integrated circuit (ASIC), or the like.

The pre-processor 110 may pre-process time series data. The time series data may be a set of data that are assembled over even periods in time and are ordered chronologically. The time series data may include at least one feature corresponding to each of a plurality of times listed chronologically. For example, the time series data may include time series medical data, which are generated by diagnosis, treatment, or medication prescription at a medical institution and represent user's health status, such as an electronic medical record (EMR). For clarity of description, time series medical data are described as an example, but a kind of the time series data is not limited thereto. For example, the time series data may be generated in various fields such as entertainment, retail, and smart management.

The pre-processor 110 may pre-process time series data such that time series irregularity of time series data, feature irregularity, and a type difference between features are corrected. The time series irregularity means that time periods between a plurality of data included in time series data are irregular. The feature irregularity means that some of a plurality of data included in time series data are missing. The feature irregularity may appear due to missing values of time series data. The type difference between features means that a criterion for generating a value differs for each feature. The pre-processor 110 may predict various measurement period points in time through the measurement period division and the modeling of the development of changes, which are associated with time series data. The pre-processor 110 may remove or supplement a missing value with respect to time series data. An operation of the pre-processor 110 will be described in detail with reference to the following drawings.

The learner 120 may train a feature model 103 based on pre-processed time series data, that is, pre-processing data. The feature model 103 may include a time series analysis model that analyzes the pre-processed time series data to calculate a future prediction result and provides prediction grounds through a prediction result. In an embodiment, the feature model 103 may be built through an artificial neural network, deep learning, or machine learning. To this end, the time series data processing device 100 may receive training data or time series data for learning from a learning database 101. The learning database 101 may be organized in a server or a storage medium that is placed outside or inside the time series data processing device 100. The learning database 101 may be organized and stored for time series management and grouping. The pre-processor 110 may pre-process time series data received from the learning database 101 and may provide the pre-processed data to the learner 120. The pre-processor 110 may perform a pre-processing operation for the purpose of interpolating or replacing feature irregularity of the time series data from the learning database 101 or generating a variety of information for processing time series irregularity of the time series data.

The learner 120 may analyze the pre-processed time series data to generate and adjust a weight group of the feature model 103. A weight group may be a set of all parameters included in a neural network structure of a feature distribution model or a neural network. The feature model 103 may be organized in a server or a storage medium that is placed outside or inside the time series data processing device 100. The weight group and the feature distribution model may be organized and stored for management.

The predictor 130 may analyze the pre-processed time series data to generate a prediction result. The prediction result may refer to a result corresponding to a prediction time such as a specific future time. To this end, the time series data processing device 100 may receive time series data for prediction and information about a prediction time from a target database 102. The target database 102 may be organized in a server or a storage medium that is placed outside or inside the time series data processing device 100. The pre-processor 110 may pre-process target data in the target database 102 so as to be provided to the predictor 130. The pre-processor 110 may perform a pre-processing operation such that time series irregularity or feature irregularity of the target data in the target database 102 is supplemented.

The predictor 130 may analyze the pre-processed time series data based on the feature model 103 trained by the learner 120. The predictor 130 may generate a prediction result 104 and prediction grounds 105 by performing analysis or machine learning on the pre-processed time series data by using the feature model 103. The prediction result 104 and the prediction grounds 105 may be organized in a server or a storage medium that is placed outside or inside the time series data processing device 100.

In an embodiment, to describe embodiments of the present disclosure easily, the learner 120 and the predictor 130 are independently described, but the present disclosure is not limited thereto. For example, the learner 120 and the predictor 130 may perform the above learning or prediction operation by using the same computational layers. That is, the learner 120 and the predictor 130 may share the same computational layer. Alternatively, the learner 120 and the predictor 130 may be implemented with the same hardware device. Alternatively, the learner 120 and the predictor 130 may perform the learning operation and the prediction operation in parallel or at the same time.

As described above, according to embodiments of the present disclosure, the time series data processing device 100 may predict various measurement period points in time with respect to time series data with time series irregularity and feature irregularity, through measurement period division and change development modeling. Accordingly, in a data environment in which measurement period information is insufficient, the time series data processing device 100 may provide an accurate prediction result and accurate prediction grounds associated with a prediction time that the user wants.

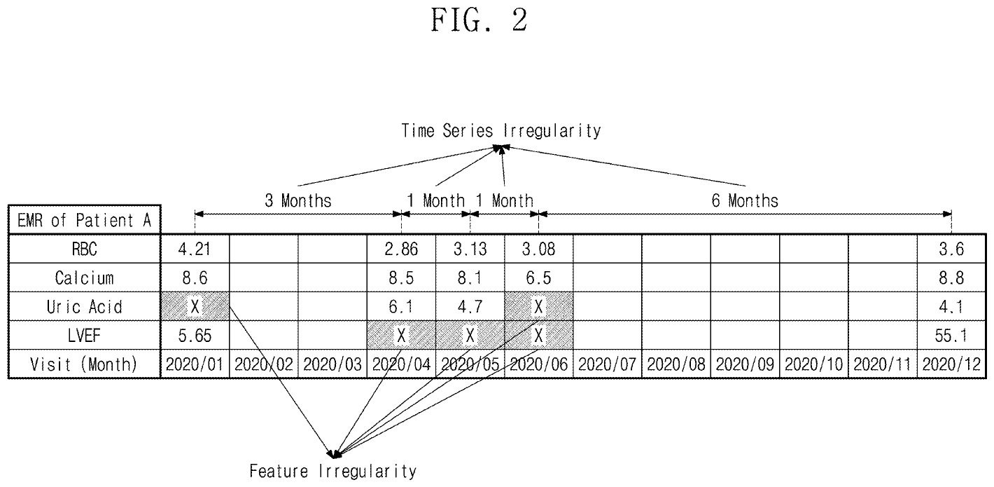

FIG. 2 is a diagram for describing time series irregularity and feature irregularity of time series data described with reference to FIG. 1. Medical time series data of a first patient are illustrated in FIG. 2 as an example. Time series data may include features of the first patient such as a red blood cell count, calcium, uric acid, and ejection fraction.

Referring to FIGS. 1 and 2, a patient may irregularly visit a hospital or a medical examination center for medical examination (or checkup). That is, time periods between time series data may be different. Also, an examination item or measured data may differ whenever the patient visits the hospital or the medical examination center.

For example, as illustrated in FIG. 2, the first patient visited a hospital in January 2020, April 2020, May 2020, June 2020, and December 2020, and measured a red blood cell count, calcium, uric acid, and ejection fraction. In this case, the visit period of the first patient may be irregular at periods of 3 months, 1 month, 1 month, and 6 months. This means time series irregularity of time series data. When the first patient visited the hospital in January 2020 and June 2020, uric acid was not measured, and when the first patient visited the hospital in April 2020, May 2020, and June 2020, ejection fraction was not measured. That is, in the time series data for January 2020 and June 2020, data associated with uric acid are missing, and in the time series data for April 2020, May 2020, and June 2020, data associated with ejection fraction are missing. The missing values may mean irregularity of time series data.

According to the general time series analysis, a prediction time may be automatically set depending on a regular time period under the assumption that a time period is regular, like data that are collected at regular periods. This analysis may fail to consider an irregular time period. In contrast, the time series data processing device 100 of FIG. 1 according to an embodiment of the present disclosure may provide a distinct prediction time in consideration of irregular time periods and may perform learning and prediction. This will be described in detail later.

As described above, a prediction result at a specific future time may not be accurate due to time series irregularity and feature irregularity of time series data. Also, time series irregularity is trained through time series data collected in a real environment (e.g., a real medical treatment environment) where the time series data are measured or collected, the accuracy of prediction may decrease. Also, because prediction grounds for a prediction process are not provided, it may be difficult to determine the reliability or validity for a prediction result.

The time series data processing device 100 according to an embodiment of the present disclosure may predict various measurement period points in time with respect to time series data with time series irregularity and feature irregularity, through measurement period division and change development modeling. Accordingly, in a data environment in which measurement period information is insufficient, the time series data processing device 100 may provide an accurate prediction result and accurate prediction grounds associated with a prediction time that the user wants.

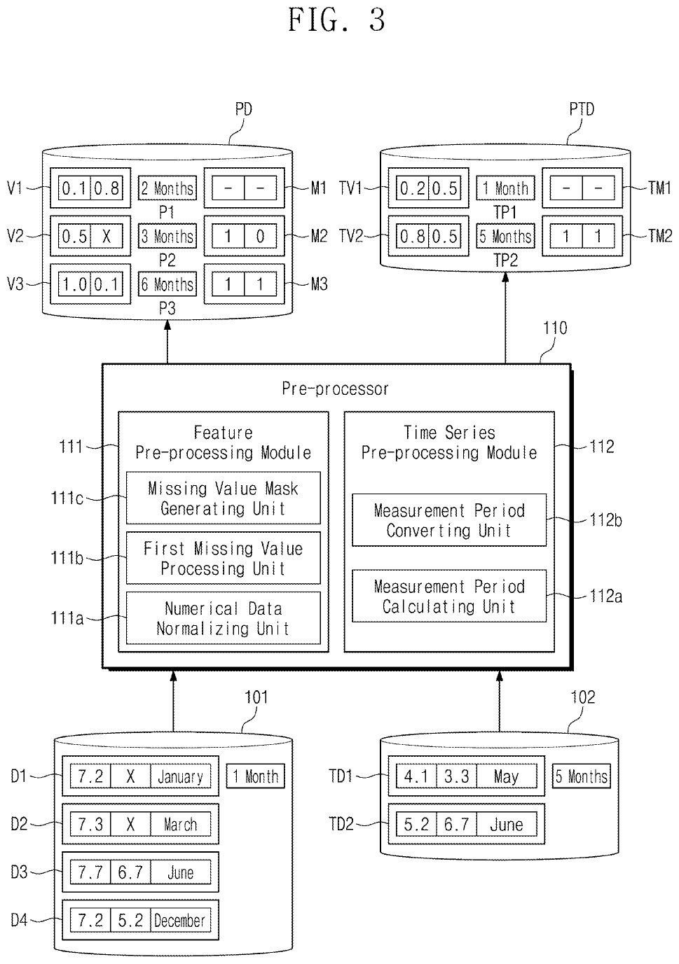

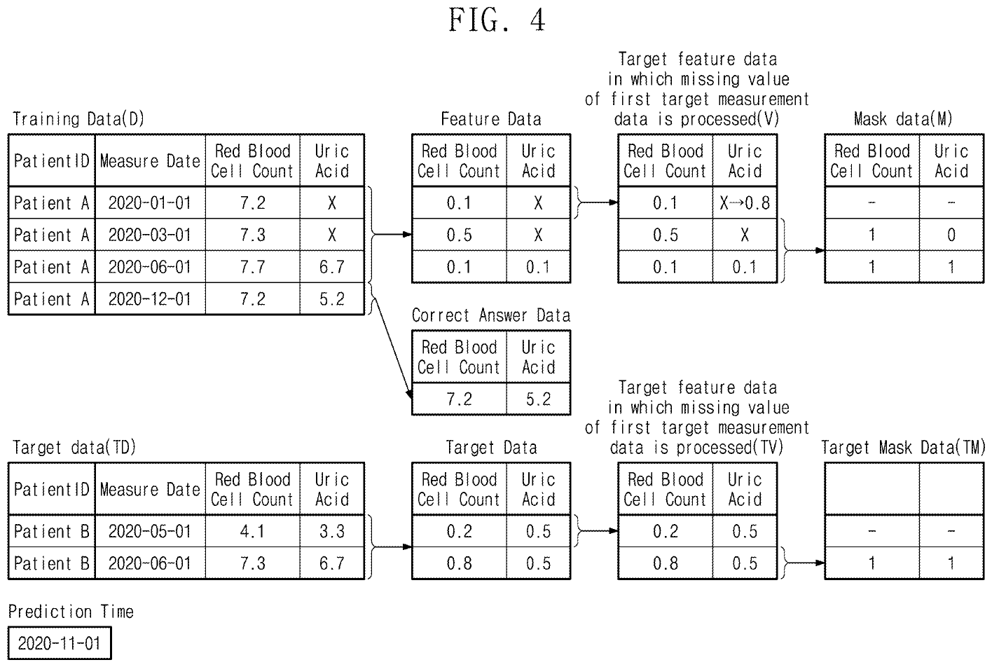

FIG. 3 is a block diagram illustrating a pre-processor of FIG. 1. FIGS. 4 and 5 are diagrams for describing a pre-processing operation of a pre-processor of FIG. 3. Below, for brevity of drawing and convenience of description, it is assumed that time series data include some features. However, the present disclosure is not limited thereto. For example, the time series data may further include various features. In the following embodiments, some numerical values are used. However, the numerical values are used to describe embodiments of the present disclosure easily, and the present disclosure is not limited thereto.

Referring to FIGS. 1 and 3, the pre-processor 110 may include a feature pre-processing module 111 and a time series pre-processing module 112. The feature pre-processing module 111 may be configured to normalize numerical values of training data and target data, to process a missing value(s), and to generate mask data for processing the missing value(s). For example, the feature pre-processing module 111 may include a numerical data normalizing unit 111a, a first missing value processing unit 111b, and a missing value mask generating unit 111c.

The numerical data normalizing unit 111a may perform normalization on a plurality of training data D1 to D4 included in the learning database 101. For example, the plurality of training data D1 to D4 may include different feature values. Different feature values may have different numerical value ranges. The numerical data normalizing unit 111a may perform a normalization operation such that the feature values of the plurality of training data D1 to D4 have the same numerical range. In an embodiment, the numerical data normalizing unit 111a may not perform the normalization operation on data corresponding to the last time from among the plurality of training data D1 to D4. The reason is as follows. Because model parameters are adjusted through the comparison between a predicted value and a real value in the process of training the feature model 103, the normalization operation may not be performed on the corresponding data for the purpose of maintaining a real value of the corresponding data.

In detail, referring to FIG, 4, training data “D” may include information about a red blood cell count and uric acid of a first patient (patient A). For brevity of drawing, a missing value is expressed by a reference sign of “X”. First training data D1 may include information about a red blood cell count and uric acid measured on Jan. 1, 2020. That is, the first training data D1 may correspond to [7.2,X]. Second training data D2 may include information about a red blood cell count and uric acid measured on Mar. 1, 2020. That is, the second training data D2 may correspond to [7.3,X]. Third training data D3 may include information about a red blood cell count and uric acid measured on Jun. 1, 2020. That is, the third training data D3 may correspond to [7.7,6.7]. Fourth training data D4 may include information about a red blood cell count and uric acid measured on Dec. 1, 2020. That is, the fourth training data D4 may correspond to [7.2,5.2]. The numerical data normalizing unit 111a may extract feature data from the training data “D” and may perform the normalization operation on the extracted feature data. The first to third training data D1 to D3 on which the normalization operation is performed may be [0.1,X], [0.5,X], and [0.1,0.1]. In an embodiment, the fourth training data D4 may be correct answer data, and the normalization operation may not be performed on the fourth training data D4.

In an embodiment, the numerical data normalizing unit 111a may perform the normalization operation on a plurality of target data TD1 and TD2 from the target database 102. As illustrated in FIG. 4, target data TD may include information about a red blood cell count and uric acid of a second patient (patient B). For example, the first target data TD1 may include information about a red blood cell count and uric acid measured on May 1, 2020. That is, the first target data TD1 may correspond to [4.1,3.3]. The second target data TD2 may include information about a red blood cell count and uric acid measured on Jun. 1, 2020. That is, the second target data TD2 may correspond to [7.3,6.7]. The numerical data normalizing unit 111a may extract feature data from the target data TD and may perform the normalization operation on the extracted feature data. The first and second target data TD1 and TD2 on which the normalization operation is performed may be [0.2,0.5] and [0.8,0.5].

The first missing value processing unit 111b of the feature pre-processing module 111 may be configured to replace or supplement the first missing value of the training data normalized by the numerical data normalizing unit 111a. For example, in the case where the first training data (or the first measurement data) of the training data include a missing value, a feature model may fail to be normally trained. Accordingly, the first missing value processing unit 111b is configured to replace the first training data of training data or a missing value of the first measurement value with a specific value. In an embodiment, the specific value may be calculated through at least one of various numerical analysis methods and may include, for example, a value corresponding to next visit data of training data, a value based on a statistical method (e.g., an average value, a median value, a central value, a maximum value, or a minimum value), a value based on a machine learning technique.

In detail, as illustrated in FIG. 4, the first training data D1 of the first to third training data D1 to D3 on which the normalization operation is performed may be the first training data (i.e., the first visit data of the first patient (patient A)). In this case, a value corresponding to uric acid of the first training data D1 experiencing the normalization operation may be a missing value. The first missing value processing unit 111b may replace the missing value (i.e., a value corresponding to uric acid) of the first training data D1 with a specific value (e.g., 0.8). In an embodiment, as described above, the specific value of 0.8 may be obtained based on at least one of various numerical analysis methods. In an embodiment, missing values present in the remaining training data other than the first training data may be decided or replaced based on a value that is predicted in the process of training a feature model.

In an embodiment, the training data “D” and the target data TD processed by the numerical data normalizing unit 111a and the first missing value processing unit 111b may be referred to as “feature data V” and “target feature data TV”. That is, first feature data V1 that are data obtained after the numerical data normalizing unit 111a and the first missing value processing unit 111b process the first training data D1 may have a value of [0.1,0.8]. Second feature data V2 that are data obtained after the numerical data normalizing unit 111a and the first missing value processing unit 111b process the second training data D2 may have a value of [0.5,X]. Third feature data V3 that are data obtained after the numerical data normalizing unit 111a and the first missing value processing unit 111b process the third training data D3 may have a value of [1.0,0.1]. First target feature data TV1 that are data obtained after the numerical data normalizing unit 111a and the first missing value processing unit 111b process the first target data TD1 may have a value of [0.2,0.5]. Second target feature data TV2 that are data obtained after the numerical data normalizing unit 111a and the first missing value processing unit 111b process the second target data TD2 may have a value of [0.8,0.5].

The missing value mask generating unit 111c of the feature pre-processing module 111 may generate mask data “M” corresponding to a missing value of the feature data “V” and the target feature data TV. For example, as illustrated in FIG. 4, the missing value mask generating unit 111c may generate the mask data “M” corresponding to the feature data “V” processed by the first missing value processing unit 111b. In this case, in the mask data “M”, a missing part of the feature data “V” may be set to “0”, and a part not missing in the feature data “V” (i.e., a measured part of the feature data “V”) may be set to “1”. In an embodiment, because a missing value of the first feature data V1 corresponding to the first training data D1 is replaced by the first missing value processing unit 111b, the generation of mask data for the first feature data V1 may be omitted. The missing value mask generating unit 111c may generate target mask data TM for the target feature data TV in the same manner, and thus, additional description will be omitted to avoid redundancy.

In an embodiment, the mask data “M” generated by the missing value mask generating unit 111c may include first to third mask data M1 to M3 respectively corresponding to the first to third feature data V1 to V3. As described above, the first mask data M1 may not be separately generated or may be set to a null value. The second mask data M2 may have a value of [1,0]. This value may indicate that a second value (i.e., a value corresponding to uric acid) of the second feature data V2 misses. The third mask data M3 may have a value of [1,1]. This value may indicate that a missing value is absent in the third feature data V3. The target mask data TM generated by the missing value mask generating unit 111c may include first and second target mask data TM1 and TM2 respectively corresponding to the first and second target feature data TV1 and TV2. As described above, the first target mask data TM1 may not be separately generated or may be set to a null value. The second target mask data TM2 may have a value of [1,1]. This value may indicate that a missing value is absent in the second target feature data TV2.

As described above, to supplement the feature irregularity of the training data “D” or the target data TD or to train a feature model, the feature pre-processing module 111 may generate the feature data “V”, the mask data “M”, the target feature data TV, and the target mask data TM by performing the following on the training data “D” or the target data TD: an operation of normalizing a numerical value, an operation of processing the first missing value, or an operation of generating mask data.

The time series pre-processing module 112 may be configured to calculate and convert a measurement period of the training data “D” or the target data TD for the purpose of allowing the feature model to learn the time series irregularity of the training data “D” or the target data TD. For example, the time series pre-processing module 112 may include a measurement period calculating unit 112a and a measurement period converting unit 112b.

The measurement period calculating unit 112a may be configured to calculate a measurement period of the training data “D” or a measurement period of the target data TD. For example, as illustrated in FIG. 5, the training data “D” may include the first to fourth training data D1 to D4 associated with the first patient (patient A). A measurement period of the first and second training data D1 and D2 may be 2 months, and a measurement period of the second and third training data D2 and D3 may be 3 months, and a measurement period of the third and fourth training data D3 and D4 may be 6 months. The measurement period calculating unit 112a may be configured to calculate measurement periods “P” between the first to fourth training data D1 to D4. The target data TD may include the first and second target data TD1 to TD2 associated with the second patient (patient B). A measurement period of the first and second target data TD1 and TD2 may be 1 month, and a period between a measurement time point of the second target data TD2 and a prediction time point may be 5 months. The measurement period calculating unit 112a may be configured to calculate a period between the first and second target data TD1 and TD2 and a period between the target data TD2 and the prediction time as a target measurement period TP.

The measurement period converting unit 112b may be configured to convert the measurement period calculated by the measurement period calculating unit 112a in a minimum unit. For example, the learning database 101 may include information about a minimum unit of a measurement period. For example, as illustrated in FIG. 3, the minimum unit of the measurement period included in the learning database 101 may be 1 month. The measurement period converting unit 112b may convert the measurement period calculated by the measurement period calculating unit 112a so as to be suitable for the minimum unit.

Measurement periods P1, P2, P3, TP1, and TP2 pre-processed by the measurement period calculating unit 112a and the measurement period converting unit 112b may correspond to the plurality of feature data V1, V2, and V3 and the plurality of target feature data TV1 and TV2. For example, the first measurement period P1 may be 2 months and may correspond to the first feature data V1. The second measurement period P2 may be 3 months and may correspond to the second feature data V2. The third measurement period P3 may be 6 months and may correspond to the third feature data V3. The first target measurement period TP1 may be 1 month and may correspond to the first target feature data TV1. The second target measurement period TP2 may be 5 months and may correspond to the second target feature data TV2.

As described above, the time series pre-processing module 112 may be configured to calculate a measurement period of the training data “D” or the target data TD for the purpose of allowing the feature model to learn the time series irregularity of the training data “D” or the target data TD.

As described above, the pre-processor 110 may pre-process the training data “D” or the target data TD and may generate pre-processed training data PD or pre-processed target data PTD. The pre-processed training data PD may include the first to third feature data V1, V2, and V3, the first to third measurement periods P1, P2, and P3, and the first to third mask data M1, M2, and M3. The pre-processed target data PTD may include the first and second target feature data TV1 and TV2, the first and second target measurement periods TP1 and TP2, and the first and second target mask data TM1 and TM2. The data or information included in the data PD and PTD is described above, and thus, additional description will be omitted to avoid redundancy. In an embodiment, the number of numerical values or data described above is an example for describing embodiments of the present disclosure easily, and the present disclosure is not limited thereto. The number of data or information or numerical values may be variously changed or modified.

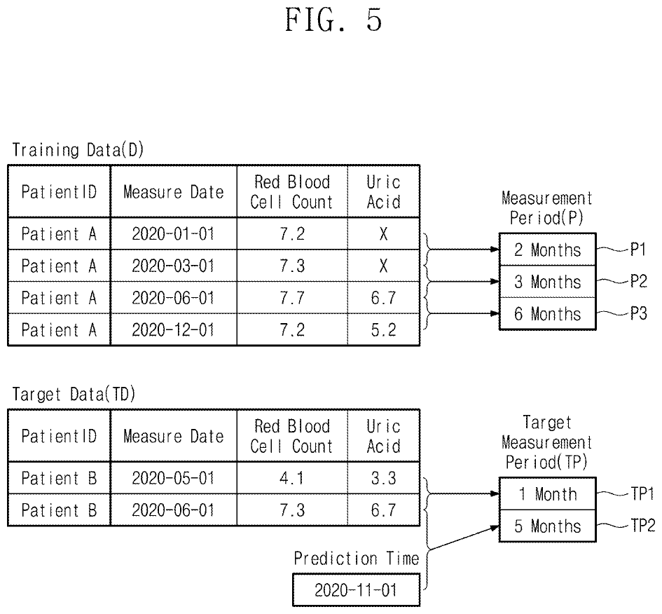

FIG. 6 is a block diagram illustrating a learner of FIG. 1. Referring to FIGS. 1 and 6, the learner 120 may be configured to perform machine learning based on the pre-processed training data PD generated by the pre-processor 110 and to create or update the feature model 103. The learner 120 may include a time series irregularity learning module 121, a feature irregularity learning module 122, and a ground tracking learning module 123.

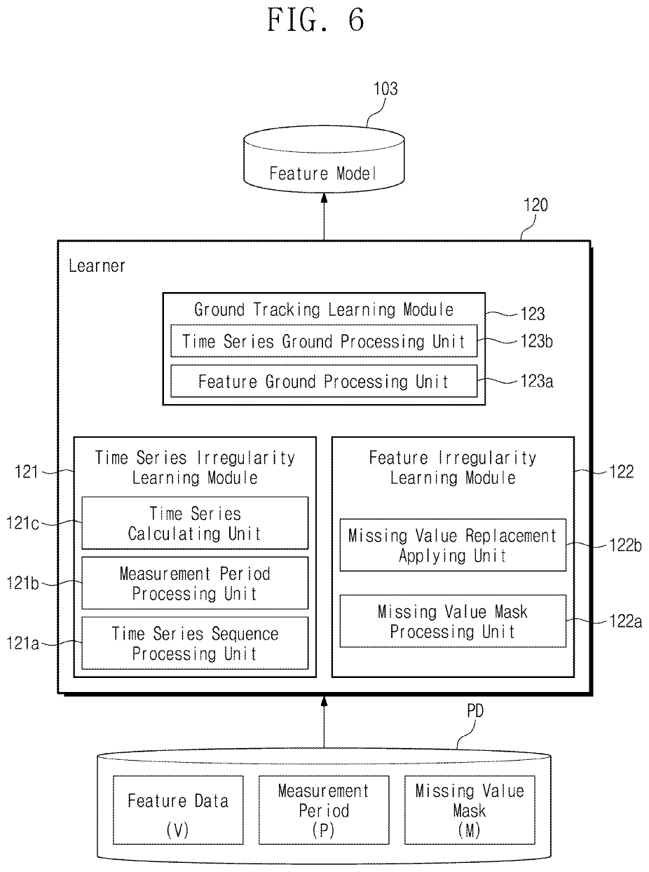

The time series irregularity learning module 121 may perform machine learning such that the feature model 103 predicts a future value at the measurement period “P” included in the pre-processed training data PD. For example, the time series irregularity learning module 121 may include a time series sequence processing unit 121a, a measurement period processing unit 121b, and a time series calculating unit 121c. The time series sequence processing unit 121a may be configured to embed the feature data “V” of the pre-processed training data PD depending on a time series sequence. The measurement period processing unit 121b may be configured to divide the measurement period “P” of the pre-processed training data PD into sub periods. The time series calculating unit 121c may be configured to calculate a prediction value appropriate for the sub period generated by the measurement period processing unit 121b, based on the feature data (i.e., embedding data) embedded by the time series sequence processing unit 121a. The time series irregularity learning module 121 may allow the feature model 103 to learn the time series irregularity of the training data “D”, based on the operations of the above components. The time series irregularity learning module 121 will be described in detail with reference to FIGS. 7 to 9.

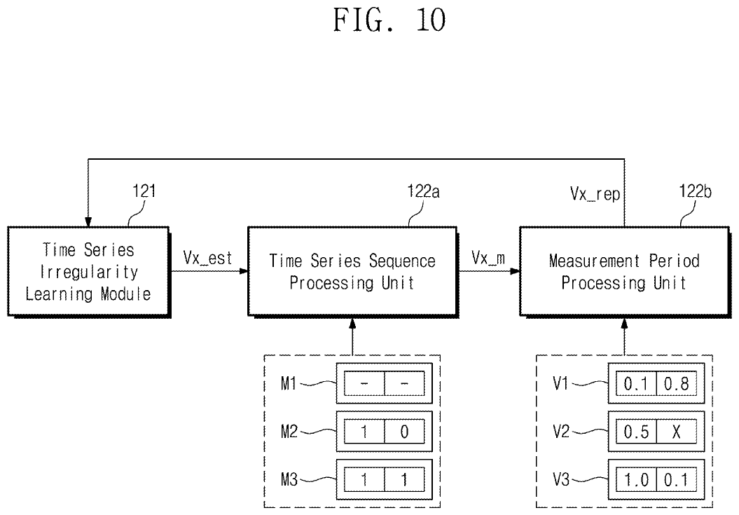

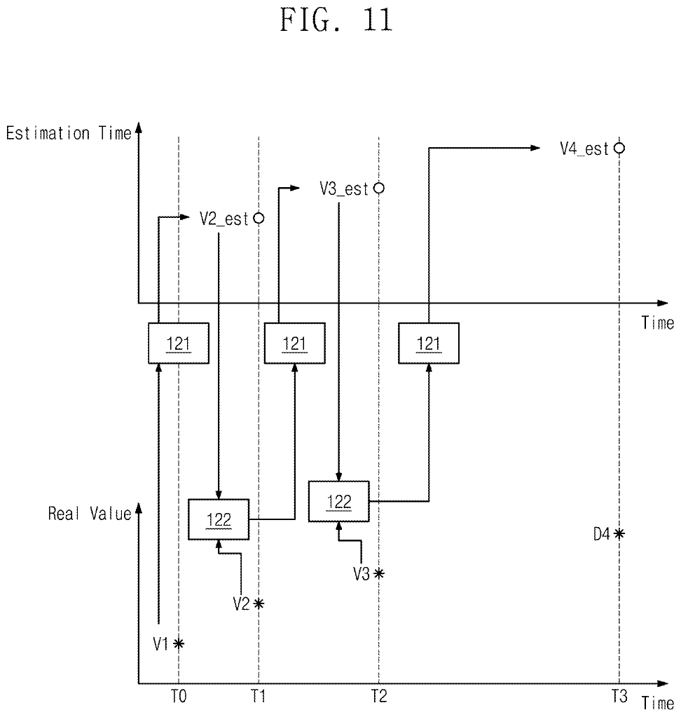

The feature irregularity learning module 122 may be configured to process a missing value included in the pre-processed training data PD. For example, the feature irregularity learning module 122 may include a missing value mask processing unit 122a and a missing value replacement applying unit 122b. The missing value mask processing unit 122a may generate missing value replacement data based on a calculation result of the time series irregularity learning module 121 and the missing value mask data “M”. The missing value replacement applying unit 122b may output feature data in which a missing value is replaced, by replacing or supplementing the missing value of the feature data “V” based on the missing value replacement data from the missing value mask processing unit 122a. In an embodiment, the feature data in which the missing value is replaced may be provided to the time series irregularity learning module 121, and the time series irregularity learning module 121 may repeatedly perform the above operation. In an embodiment, the operation of the feature irregularity learning module 122 will be described in detail with reference to FIGS. 10 and 11.

The ground tracking learning module 123 may provide prediction grounds associated with the prediction result. For example, the ground tracking learning module 123 may include a feature ground processing unit 123a configured to provide feature grounds, and a time series ground processing unit 123b configured to provide time series grounds.

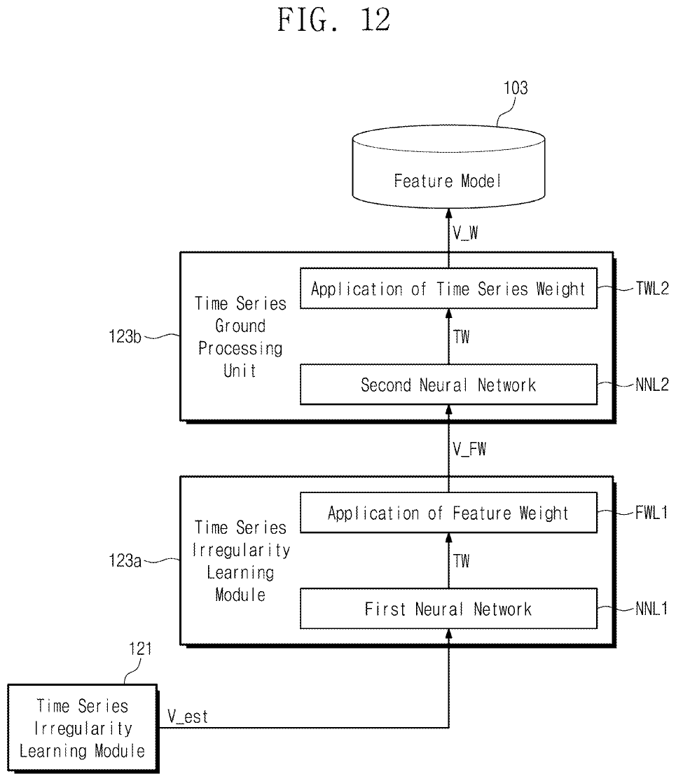

In an embodiment, the prediction grounds may refer to information or data for describing how a result predicted by the feature model 103 is calculated. For example, in the case of predicting a future value or a disease by using medical data being an example of time series data, the prediction grounds may be important. Because there occurs the case where the accuracy of the feature model 103 is low or incorrect, the prediction grounds describing the process of calculating a prediction value is essentially required to determine whether the prediction by the feature model 103 is accurate. To this end, the ground tracking learning module 123 according to an embodiment of the present disclosure may train the feature model 103 such that feature grounds and time series grounds are drawn. In an embodiment, an operation and a configuration of the ground tracking learning module 123 will be described in detail with reference to FIG. 12.

Below, components will be separately described to describe an operation and a configuration of the learner 120 according to an embodiment of the present disclosure. However, the present disclosure is not limited thereto. For example, it may be understood that the learner 120 may be implemented with a combination of various components to be described with reference to in the following embodiments of the learner 120.

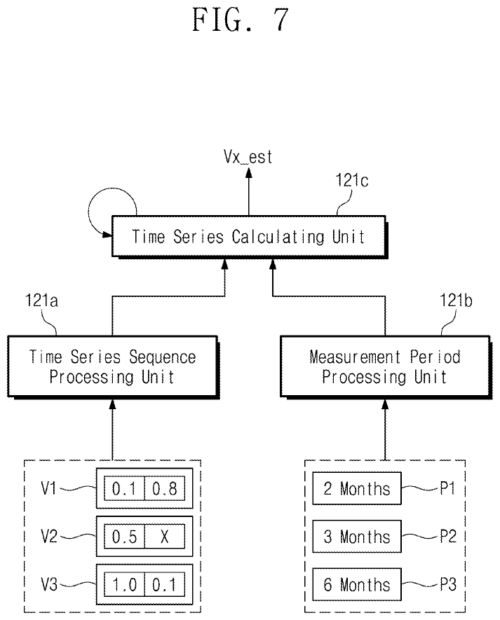

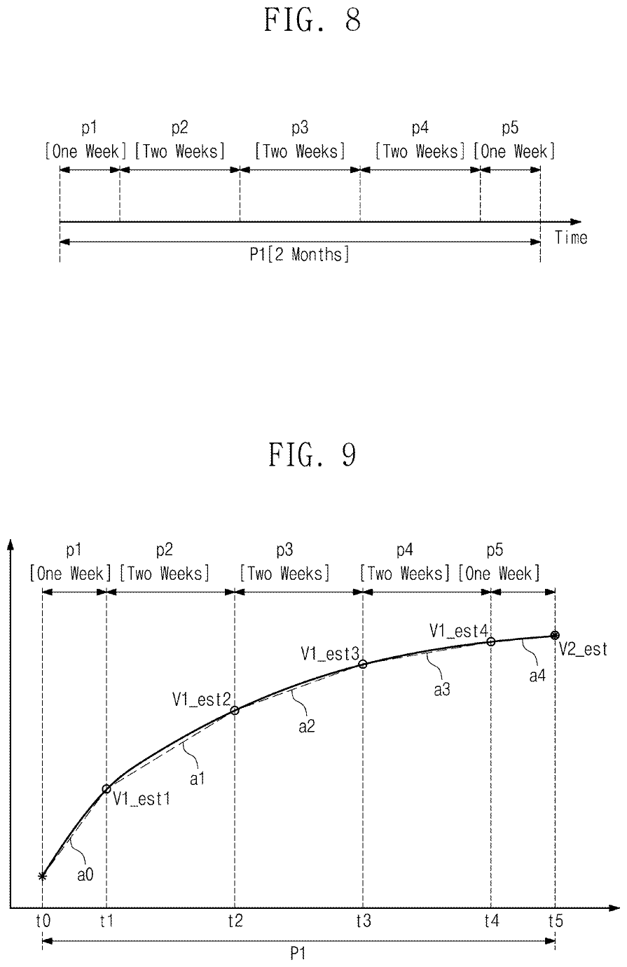

FIG. 7 is a block diagram illustrating a time series irregularity learning module of FIG. 6. FIG. 8 is a diagram for describing an operation of a measurement period processing unit of FIG. 7. FIG. 9 is a diagram for describing an operation of a time series calculating unit of FIG. 7.

Referring to FIGS. 6 to 9, the time series irregularity learning module 121 may include the time series sequence processing unit 121a, the measurement period processing unit 121b, and the time series calculating unit 121c. The time series sequence processing unit 121a may be configured to embed the plurality of feature data V1, V2, and V3 of the pre-processed training data PD depending on a time series sequence. For example, in the case where the plurality of feature data V1, V2, and V3 have a time series feature in order of the first feature data V1, the second feature data V2, and the third feature data V3, the time series sequence processing unit 121a may embed the plurality of feature data V1, V2, and V3 in order of the first feature data V1, the second feature data V2, and the third feature data V3. In an embodiment, embedding may indicate the process of quantifying a value (e.g., a sex or a doctor's note) not being a numerical value from among values included in each of the plurality of feature data V1, V2, and V3. Feature data embedded by the time series sequence processing unit 121a may be provided to the time series calculating unit 121c.

The measurement period processing unit 121b may be configured to divide the pre-processed measurement periods P1, P2, and P3 into sub periods. For example, the first measurement period P1 corresponding to the first feature data V1 may be 2 months. The measurement period processing unit 121b may divide the first measurement period P1 being 2 months into sub periods. In an embodiment, the sub periods may have arbitrary periods. As an example, as illustrated in FIG. 8, the first measurement period P1 being 2 months may be divided into first to fifth sub periods p1, p2, p3, p4, and p5. Each of the first and fifth sub periods p1 and p5 may be one week, and each of the second to fourth sub periods p2 to p4 may be two weeks. However, the present disclosure is not limited thereto. For example, a range of a sub period may be variously changed. In an embodiment, why to divide each of the measurement periods P1, P2, and P3 being the pre-processed training data PD into arbitrary sub periods may be for training the feature model 103 by using various measurement periods. Information about the sub periods divided by the measurement period processing unit 121b may be provided to the time series calculating unit 121c.

The time series calculating unit 121c may receive the embedded feature data from the time series sequence processing unit 121a and may receive information about an arbitrary sub period from the measurement period processing unit 121b. The time series calculating unit 121c may be configured to calculate a prediction value appropriate for the arbitrary sub period, through the embedded feature data. In an embodiment, the process of calculating a prediction value may be performed based on machine learning or a neural network.

For example, as illustrated in FIG. 9, the time series calculating unit 121c may receive the first feature data V1 from the time series sequence processing unit 121a. For convenience of description, the expression “first feature data V1” may be used, but the first feature data V1 of FIG. 9 may be data or a vector embedded by the time series sequence processing unit 121a.

The time series calculating unit 121c may be configured to predict or calculate first prediction data V1_est1, which are a prediction value after the first sub period p1 (i.e., one week), with respect to the first feature data V1. The prediction or calculation of the first prediction data V1_est1 may be performed or implemented through a neural network that estimates a function for a slope of a distribution of feature data. For example, a 0-th slope a0 between the first feature data V1 and the first prediction data V1_est1 may be expressed by a function of f(V1, p1). In this case, the time series calculating unit 121c may predict or estimate the 0-th slope a0 by using the neural network estimating the function (f). The time series calculating unit 121c may predict or calculate the first prediction data V1_est1 based on the first feature data V1, the 0-th slope a0, and the first sub period p1.

As in the above description, the time series calculating unit 121c may be configured to predict a first slope a1 between the first prediction data V1_est1 and a second prediction data V1_est2, which is a prediction value after the second sub period p2 (i.e., two weeks), based on the function (f), and to predict or calculate the second prediction data V1_est2 with respect to the first prediction data V1_est1 based on the first slope a1. The time series calculating unit 121c may be configured to predict a second slope a2 between the second prediction data V1_est2 and a third prediction data V1_est3, which is a prediction value after the third sub period p3 (i.e., two weeks), based on the function (f), and to predict or calculate the third prediction data V1_est3 with respect to the second prediction data V1_est2 based on the second slope a2. The time series calculating unit 121c may be configured to predict a third slope a3 between the third prediction data V1_est3 and a fourth prediction data V1_est4, which is a prediction value after the fourth sub period p4 (i.e., two weeks), based on the function (f), and to predict or calculate the fourth prediction data V1_est4 with respect to the third prediction data V1_est3 based on the third slope a3. The time series calculating unit 121c may be configured to predict a fourth slope a4 between the fourth prediction data V1_est4 and a prediction data V2_est of the second feature data V2, which is a prediction value after the fifth sub period p5 (i.e., one weeks), based on the function (f), and to predict or calculate the prediction data V2_est of the second feature data V2 with respect to the fourth prediction data V1_est4 based on the fourth slope a4. The above prediction processes are similar to the above process of predicting or calculating the first prediction data V1_est1, and thus, additional description will be omitted to avoid redundancy.

As described above, the time series irregularity learning module 121 may be configured to calculate a prediction value after an arbitrary sub period with respect to each of the plurality of feature data V1, V2, and V3 of the pre-processed training data PD.

In an embodiment, the time series irregularity learning module 121 may operate as described above, with regard to the first measurement data of the pre-processed training data PD; the time series irregularity learning module 121 may perform the above operation with regard to the following measurement data, based on replacement data that are generated by the feature irregularity learning module 122, which will be described below.

FIG. 10 is a block diagram illustrating a feature irregularity learning module of FIG. 6. Referring to FIGS. 6 and 10, the feature irregularity learning module 122 may include the missing value mask processing unit 122a and the missing value replacement applying unit 122b.

The missing value mask processing unit 122a may be configured to generate prediction data Vx_m masked by using the mask data “M” from among the pre-processed training data PD. For example, through the operation described with reference to FIGS. 7 to 9, the time series irregularity learning module 121 may output prediction data (i.e., the prediction data V2_est associated with the second feature data V2) after the first measurement period P1 (i.e., 2 months) passes, with regard to the first feature data V1 of the pre-processed training data PD. In an embodiment, to express the iterative operation between the time series irregularity learning module 121 and the feature irregularity learning module 122, prediction data output from the time series irregularity learning module 121 are marked by a reference sign of “Vx_est” in FIG. 10. Herein, “x” indicates a number for referring to corresponding prediction data or feature data.

The missing value mask processing unit 122a may generate second masked prediction data V2_m based on the second mask data M2, that is, [1,0]. For example, with regard to the second feature data V2, it is assumed that the prediction data V2_est predicted by the time series irregularity learning module 121 are [0.4,0.1] and the second mask data M2 are [1,0]. As described with reference to FIGS. 3 and 4, that a value of mask data is “0” means that the value corresponds to a missing value. Accordingly, the missing value mask processing unit 122a may output the masked prediction data V2_m of [x,0.1] based on the prediction data V2_est and the second mask data M2.

The missing value replacement applying unit 122b may generate replacement data Vx_rep by applying the masked prediction data Vx_m to the feature data V of the pre-processed training data PD. For example, as described above, in the case where the masked prediction data V2_m are [x,0.1] and the second feature data V2 are [0.5,X], the second replacement data V2_rep may be [0.5,0.1]. The replacement data Vx_rep generated by the missing value replacement applying unit 122b may be provided to the time series irregularity learning module 121. The time series irregularity learning module 121 may perform the prediction operation, which is described with reference to FIGS. 7 to 9, by using the replacement data Vx_rep.

As described above, the feature irregularity learning module 122 may generate replacement data by replacing a value of feature data, which corresponds to a missing value, with a value predicted by the time series irregularity learning module 121. Accordingly, even though a missing value is included in the training data “D” or the pre-processed training data PD, because the missing value is replaced or supplemented through the feature irregularity learning module 122, the feature irregularity may be solved, or the feature model 103 may learn the feature irregularity.

FIG. 11 is a diagram for describing operations of a time series irregularity learning module and a feature irregularity learning module of FIG. 6. In an embodiment, the operation of the time series irregularity learning module 121 described with reference to FIGS. 7 to 9 and the operation of the feature irregularity learning module 122 described with reference to FIG. 10 may be organically or repeatedly performed. For example, referring to FIGS. 6 and 11, the pre-processed training data PD may include the first to fourth feature data V1, V2, V3, and V4. The first to fourth feature data V1, V2, V3, and V4 may correspond to real data measured from the first patient (patient A) at a first time t1, a second time t2, a third time t3, and a fourth time t4, respectively.

The time series irregularity learning module 121 may perform the machine learning or the neural network on the first feature data V1 and may generate the prediction data V2_est associated with the second feature data V2. The prediction data V2_est predicted by the time series irregularity learning module 121 may be provided to the feature irregularity learning module 122. The feature irregularity learning module 122 may replace or supplement a missing value of the second feature data V2 by using the prediction data V2_est. The replacement data in which the missing value is replaced by the feature irregularity learning module 122 may be provided to the time series irregularity learning module 121.

The time series irregularity learning module 121 may perform the machine learning or the neural network on the replacement data received from the feature irregularity learning module 122 and may generate the prediction data V3_est associated with the third feature data V3. The prediction data V3_est predicted by the time series irregularity learning module 121 may be provided to the feature irregularity learning module 122. The feature irregularity learning module 122 may replace or supplement a missing value of the third feature data V3 by using the prediction data V3_est. The replacement data in which the missing value is replaced by the feature irregularity learning module 122 may be provided to the time series irregularity learning module 121.

The time series irregularity learning module 121 may perform the machine learning or the neural network on the replacement data received from the feature irregularity learning module 122 and may generate the prediction data V4_est associated with the fourth feature data V4.

In an embodiment, each of the prediction operations of the time series irregularity learning module 121 may be performed based on the operation described with reference to FIGS. 7 to 9. That is, to calculate one prediction data, the neural network or calculation may be performed for each of a plurality of sub periods.

FIG. 12 is a block diagram illustrating a ground tracking learning module of FIG. 6. Referring to FIGS. 6 and 12, the ground tracking learning module 123 may be configured to draw prediction grounds associated with a prediction result. For example, the ground tracking learning module 123 may include the feature ground processing unit 123a and the time series ground processing unit 123b.

The feature ground processing unit 123a may perform the neural network operation on the prediction data V_est associated with all the times predicted by the time series irregularity learning module 121. For example, the feature ground processing unit 123a may perform the neural network operation on the prediction data V_est through a first neural network NNL1 and may decide a feature weight FW. In an embodiment, the feature weight FW may refer to a weight according to a correlation between pieces of feature data (or check items) that are used to draw final prediction data. For example, in the case of generating a feature model that predicts a numerical value associated with a red blood cell count after 5 months, the first neural network NNL1 may be a neural network that allows a high weight to be applied to feature data having high correlation with the red blood cell count. In an embodiment, the first neural network NNL1 may be a neural network of an attention mechanism.

The feature ground processing unit 123a may apply the feature weight FW generated by the first neural network NNL1 to the prediction data V_est and may output feature data V_FW to which the feature weight FW is applied. This may be performed by a feature weight applying layer FWL1.

The time series ground processing unit 123b may be configured to generate a time series weight TW by using a second neural network NNL2. A time series weight refers a weight according to a correlation between visit times that are used to draw final prediction data. For example, in the case of generating a feature model that predicts a numerical value associated with a red blood cell count after 5 months, the second neural network NNL2 may determine whether feature data corresponding to any visit time from among feature data of previous visit times, which are associated with a red blood cell count, have the highest correlation. The time series ground processing unit 123b may apply the time series weight TW to the feature data V_FW to which the feature weight FW is applied and may output feature data V_W to which a final weight is applied. The feature data V_W to which the final weight is applied may be stored in the feature model 103, may be used to update weights of the feature model 103, or may provide prediction grounds.

In an embodiment, to describe an embodiment of the present disclosure easily, the ground tracking learning module 123 is described under the condition that the feature ground processing unit 123a operates and the time series ground processing unit 123b then operates. However, the present disclosure is not limited thereto. For example, the order of operating the feature ground processing unit 123a and the time series ground processing unit 123b may be exchangeable. Alternatively, the feature ground processing unit 123a and the time series ground processing unit 123b may operate at the same time or in parallel.

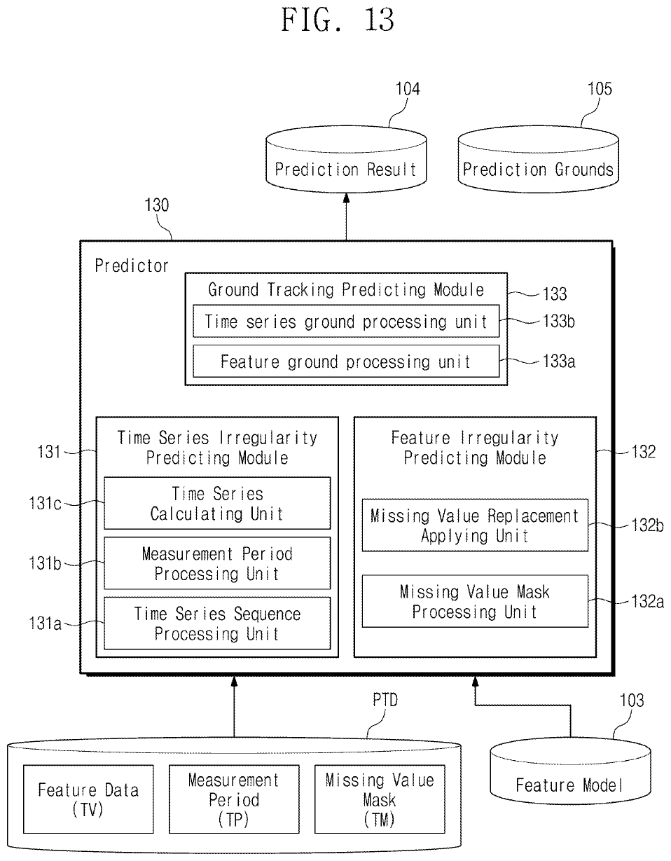

FIG. 13 is a block diagram illustrating a predictor of FIG. 1. Referring to FIGS. 1 and 13, the predictor 130 may be configured to draw final prediction data or future feature data by performing machine learning based on the pre-processed target data PTD and the feature model 103.

For example, the predictor 130 may include a time series irregularity predicting module 131, a feature irregularity predicting module 132, and a ground tracking predicting module 133. The time series irregularity predicting module 131 may include a time series sequence processing unit 131a, a measurement period processing unit 131b, and a time series calculating unit 131c. In an embodiment, an operation of the time series irregularity predicting module 131 is similar to the operation of the time series irregularity learning module 121 described with reference to FIGS. 6 to 9 except that calculation is performed based on the pre-processed target data PTD and the feature model 103, and thus, additional description will be omitted to avoid redundancy.

The feature irregularity predicting module 132 may include a missing value mask processing unit 132a and a missing value replacement applying unit 132b. The feature irregularity predicting module 132 is similar to the feature irregularity learning module 122 described with reference to FIG. 10, and thus, additional description will be omitted to avoid redundancy. In an embodiment, as in the above description given with reference to FIG. 11, the time series irregularity predicting module 131 and the feature irregularity predicting module 132 may operate organically or repeatedly. In an embodiment, the final prediction data from the time series irregularity predicting module 131 may be output or stored as the prediction result 104

The ground tracking predicting module 133 may include a feature ground processing unit 133a and a time series ground processing unit 133b. The ground tracking predicting module 133 is similar to the ground tracking learning module 123 described with reference to FIG. 12, and thus, additional description will be omitted to avoid redundancy.

As described above, the predictor 130 may be configured to apply time series irregularity and feature irregularity based on the pre-processed target data PTD and to draw the final prediction result 104 and the prediction grounds 105.

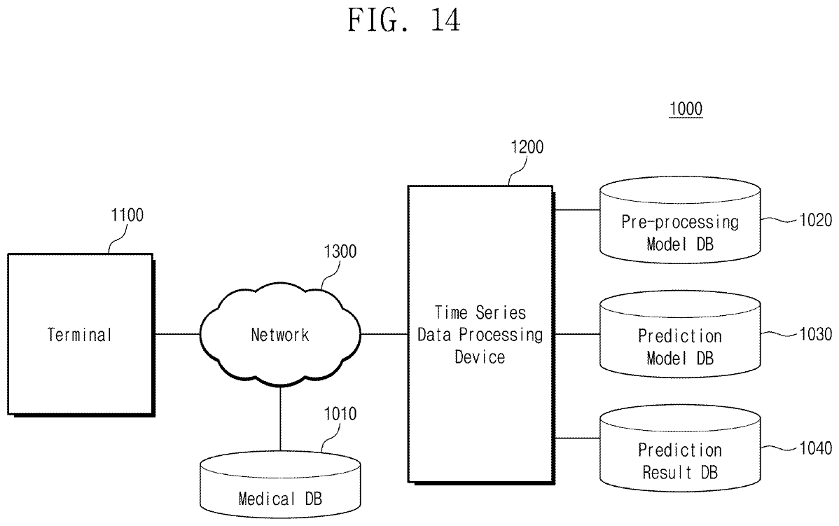

FIG. 14 is a diagram illustrating a health status predicting system to which a time series data processing device of FIG. 1 is applied. Referring to FIG. 14, a health status predicting system 1000 includes a terminal 1100, a time series data processing device 1200, and a network 1300.

The terminal 1100 may collect time series data from the user and may provide the collected time series data to the time series data processing device 1200. For example, the terminal 1100 may collect time series data from a medical database 1010 or the like. The terminal 1100 may include one of various electronic devices, which are capable of receiving time series data from the user, such as a smartphone, a desktop computer, a laptop computer, and a wearable device. The terminal 1100 may include a communication module or a network interface configured to transmit time series data over the network 1300. FIG. 14 shows one terminal 1100, but the present disclosure is not limited thereto. For example, time series data may be provided from a plurality of terminals to the time series data processing device 1200.

The medical database 1010 may be configured to integrate and manage medical data associated with various users. The medical database 1010 may include the learning database 101 or the target database 102 of FIG. 1. For example, the medical database 1010 may receive medical data from a public institution, a hospital, a user, and the like. The medical database 1010 may be implemented with a server or a storage medium. The medical data may be stored in the medical database 1010 for time series management and grouping. The medical database 1010 may periodically provide time series data to the time series data processing device 1200 over the network 1300.

The time series data may include time series medical data, which are generated by diagnosis, treatment, or medication prescription at a medical institution and represent user's health status, such as an electronic medical record (EMR). The time series data may be generated when the user visits a medical center for diagnosis, treatment, or medication prescription. The time series data may include pieces of data that are listed chronologically as the user visits the medical center. The time series data may include a plurality of features generated based on diagnosis, treatment, or medication prescription-related features. For example, a feature may include data measured by a blood pressure monitor, or data indicating the degree of a disease such as arteriosclerosis.

The time series data processing device 1200 may build a learning model through the time series data received from the medical database 1010 (or the terminal 1100). For example, the learning model may include a prediction model for predicting a future health status based on time series data. For example, the learning model may include a pre-processing model for pre-processing time series data. The time series data processing device 1200 may perform the following based on the time series data received from the medical database 1010: training the learning model and generating a weight group. To this end, the pre-processor 110 and the learner 120 of FIG. 1 may be implemented in the time series data processing device 1200.

The time series data processing device 1200 may process time series data received from the terminal 1100 or the medical database 1010 based on the built learning model. The time series data processing device 1200 may pre-process the time series data based on the built pre-processing model. The time series data processing device 1200 may analyze the pre-processed time series data based on the built prediction model. As an analysis result, the time series data processing device 1200 may calculate a prediction result corresponding to a prediction time. The prediction result may correspond to the future health status of the user. To this end, the pre-processor 110 and the predictor 130 of FIG. 1 may be implemented in the time series data processing device 1200.

A pre-processing model database 1020 is configured to integrate and manage the pre-processing model and the weight group obtained through the learning of the time series data processing device 1200. The pre-processing model database 1020 may be implemented with a server or a storage medium. For example, the pre-processing model may include a model for interpolating missing values associated with features included in the time series data.

A prediction model database 1030 is configured to integrate and manage the prediction model and the weight group obtained through the learning of the time series data processing device 1200. The prediction model database 1030 may include the feature model 103 of FIG. 1. The prediction model database 1030 may be implemented with a server or a storage medium.

A prediction result database 1040 is configured to integrate and manage the prediction result analyzed by the time series data processing device 1200. The prediction result database 1040 may include the prediction result 104 of FIG. 1. The prediction result database 1040 may be implemented with a server or a storage medium.

The network 1300 may be configured to enable the data communication between the terminal 1100, the medical database 1010, and the time series data processing device 1200. The terminal 1100, the medical database 1010, and the time series data processing device 1200 may exchange data wiredly or wirelessly over the network 1300.

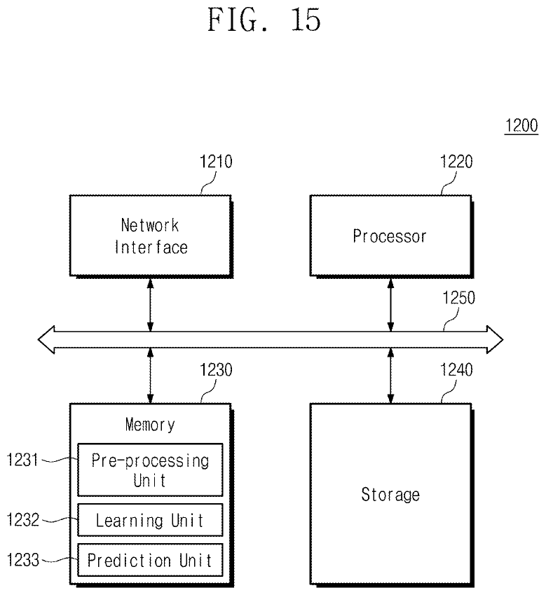

FIG. 15 is a block diagram of a time series data processing device of FIG. 1 or 14. The block diagram of FIG. 15 may be understood as an example configuration for pre-processing time series data, generating a weight group based on the pre-processed time series data, and generating a prediction result based on the weight group, and a structure of a time series data processing device may not be limited thereto. Referring to FIG. 15, the time series data processing device 1200 may include a network interface 1210, a processor 1220, a memory 1230, storage 1240, and a bus 1250. In an embodiment, the time series data processing device 1200 may be implemented with a server, but the present disclosure is not limited thereto.

The network interface 1210 may be configured to receive time series data, which are provided from the terminal 1100 or the medical database 1010, over the network 1300 of FIG. 14. The network interface 1210 may provide the received time series data to the processor 1220, the memory 1230, or the storage 1240 over the bus 1250. Also, the network interface 1210 may be configured to provide a prediction result of the future health status, which is generated based on the received time series data, to the terminal 1100 over the network 1300 of FIG. 14.

The processor 1220 may function as a central processing unit of the time series data processing device 1200. The processor 1220 may perform a control operation and a computation/calculation operation that are required to implement the pre-processing and data analysis of the time series data processing device 1200. For example, under control of the processor 1220, the network interface 1210 may receive time series data from the outside. Under control of the processor 1220, a calculation operation for generating a weight group of a prediction model may be performed, and a prediction result may be obtained by using the prediction model. The processor 1220 may operate by utilizing a computation/calculation space of the memory 1230 and may read files for driving an operating system and execution files of applications from the storage 1240. The processor 1220 may execute the operating system and the applications.

The memory 1230 may store data and program codes that are processed by the processor 1220 or are scheduled to be processed by the processor 1220. For example, the memory 1230 may store time series data, information for pre-processing the time series data, information for generating a weight group, information for calculating a prediction result, and information for building a prediction model. The memory 1230 may be used as a main memory of the time series data processing device 1200. The memory 1230 may include a dynamic random access memory (DRAM), a static RAM (SRAM), a phase-change RAM (PRAM), a magnetic RAM (MRAM), a ferroelectric RAM (FeRAM), a resistive RAM (RRAM), or the like.

A pre-processing unit 1231, a learning unit 1232, and a prediction unit 1233 may be loaded and executed onto the memory 1230. The pre-processing unit 1231, the learning unit 1232, and the prediction unit 1233 respectively correspond to the pre-processor 110, the learner 120, and the predictor 130 of FIG. 1. The pre-processing unit 1231, the learning unit 1232, and the prediction unit 1233 may be a portion of the calculation space of the memory 1230. In this case, the pre-processing unit 1231, the learning unit 1232, and the prediction unit 1233 may be implemented in the form of firmware or software. For example, the firmware may be stored in the storage 1240 and may be loaded onto the memory 1230 upon executing the firmware. The processor 1220 may execute the firmware loaded onto the memory 1230. The pre-processing unit 1231 may operate under control of the processor 1220 so as to pre-process time series data. The learning unit 1232 may operate under control of the processor 1220 so as to analyze the pre-processed time series data and to generate a weight group as an analysis result. The prediction unit 1233 may be configured to generate a prediction result based on the weight group generated under control of the processor 1220.

The storage 1240 may store data generated for the purpose of long-time storage by the operating system or the applications, files for driving the operating system, execution files of the applications, etc. For example, the storage 1240 may store files for execution of the pre-processing unit 1231, the learning unit 1232, and the prediction unit 1233. The storage 1240 may be used as an auxiliary storage device of the time series data processing device 1200. The storage 1240 may include a flash memory, a PRAM, an MRAM, a FeRAM, an RRAM, etc.

The bus 1250 may provide a communication path between the components of the time series data processing device 1200. The network interface 1210, the processor 1220, the memory 1230, and the storage 1240 may exchange data with each other over the bus 1250. The bus 1250 may be configured to support various communication formats used in the time series data processing device 1200.

According to embodiments of the present disclosure, a time series data processing device may predict various measurement period points in time with respect to time series data with time series irregularity and feature irregularity, through measurement period division and change development modeling. In this case, in a data environment where measurement period information is insufficient, the time series data processing device may provide an accurate prediction result and accurate prediction grounds associated with a prediction time that the user wants. Accordingly, there is provided the time series data processing device configured to process time series data with irregularity, the reliability of which is improved.

While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. A time series data processing device comprising:
a pre-processor configured to perform pre-processing on time series data to generate pre-processing data; and
a learner configured to create or update a feature model through machine learning for the pre-processing data,
wherein the learner includes:
a time series irregularity learning model configured to learn time series irregularity of the pre-processing data; and
a feature irregularity learning model configured to learn feature irregularity of the pre-processing data.

2. The time series data processing device of claim 1, wherein the pre-processor includes:
a numerical data normalizing unit configured to normalize the time series data to generate a plurality of feature data;
a first missing value processing unit configured to replace a missing value of first feature data of the plurality of feature data with a specific value; and
a missing value mask generating unit configured to generate mask data based on a missing value of the plurality of feature data.

3. The time series data processing device of claim 2, wherein the specific value is decided based on at least one of a value corresponding to next feature data associated with a feature corresponding to the missing value of the first feature data of the plurality of feature data, an average value, a median value, a central value, a maximum value, or a minimum value, a value based on a machine learning technique.

4. The time series data processing device of claim 2, wherein the pre-processor further includes:
a measurement period calculating unit configured to calculate a period of the time series data; and
a measurement period converting unit configured to convert the period calculated from the measurement period calculating unit into a minimum unit to output a measurement period, and
wherein the pre-processing data include the plurality of feature data, the measurement period, and the mask data.

5. The time series data processing device of claim 4, wherein the time series irregularity learning model includes:
a time series sequence processing unit configured to embed the plurality of feature data of the pre-processing data to output a plurality of embedding data;
a measurement period processing unit configured to divide the measurement period into a plurality of sub periods; and
a time series calculating unit configured to calculate a plurality of first prediction data respectively associated with the plurality of sub periods based on first embedding data of the plurality of embedding data.

6. The time series data processing device of claim 5, wherein the time series calculating unit is configured to:
estimate a first slope based on a first sub period of the plurality of sub periods and the first embedding data;
calculate one prediction data of the plurality of first prediction data based on the first slope, the first sub period, and the first embedding data;
estimate a second slope based on a second sub period of the plurality of sub periods and the one prediction data; and
calculate another prediction data of the plurality of first prediction data based on the second slope, the second sub period, and the one prediction data.

7. The time series data processing device of claim 6, wherein the first slope and the second slope are estimated based on a neural network estimating a function of a slope of a distribution of the plurality of feature data.

8. The time series data processing device of claim 5, wherein the feature irregularity learning model includes:
a missing value mask processing unit configured to generate masked prediction data based on last prediction data of the plurality of first prediction data and the mask data; and
a missing value replacement applying unit configured to generate replacement data by replacing a missing value of feature data corresponding to the masked prediction data from among the plurality of feature data, based on the masked prediction data.

9. The time series data processing device of claim 8, wherein the time series calculating unit is further configured to:
calculate a plurality of second prediction data respectively associated with the plurality of sub periods based on the replacement data.

10. The time series data processing device of claim 9, further comprising:
a feature ground processing unit configured to:
perform a first neural network operation on the plurality of first prediction data and the plurality of second prediction data to decide a feature weight; and
apply the feature weight to the plurality of first prediction data and the plurality of second prediction data to generate data to which the feature weight is applied,
wherein the feature weight indicates a correlation between the plurality of feature data.

11. The time series data processing device of claim 9, further comprising:
a time series ground processing unit configured to:
perform a second neural network operation on the plurality of first prediction data and the plurality of second prediction data to decide a time series weight; and
apply the time series weight to the plurality of first prediction data and the plurality of second prediction data to generate data to which the time series weight is applied,
wherein the time series weight indicates a correlation associated with the period of the time series data.

12. A time series data processing device comprising:
a pre-processor configured to perform pre-processing on time series data to generate pre-processing data; and
a predictor configured to perform machine learning on the pre-processing data based on a feature model and to output a prediction result and prediction grounds,
wherein the predictor includes:
a time series irregularity predicting module configured to calculate a plurality of prediction data based on the feature model and a sub period smaller than a measurement period of the pre-processing data;
a feature irregularity predicting module configured to replace a missing value of the pre-processing data based on the plurality of prediction data; and
a ground tracking predicting module configured to generate a feature weight and a time series weight based on the plurality of prediction data, to apply the feature weight and the time series weight to the plurality of prediction data, and to output data to which a weight is applied,
wherein the prediction result includes at least one of the plurality of prediction data, and the prediction grounds include the data to which the weight is applied.

13. The time series data processing device of claim 12, wherein the pre-processor includes:
a numerical data normalizing unit configured to normalize the time series data to generate a plurality of feature data;
a first missing value processing unit configured to replace a missing value of first feature data of the plurality of feature data with a specific value; and
a missing value mask generating unit configured to generate mask data based on a missing value of the plurality of feature data.

14. The time series data processing device of claim 13, wherein the pre-processor further includes:
a measurement period calculating unit configured to calculate a period of the time series data; and
a measurement period converting unit configured to convert the period calculated from the measurement period calculating unit into a minimum unit to output a measurement period, and
wherein the pre-processing data include the plurality of feature data, the measurement period, and the mask data.

15. The time series data processing device of claim 14, wherein the time series irregularity predicting model includes:
a time series sequence processing unit configured to embed the plurality of feature data of the pre-processing data to output a plurality of embedding data;
a measurement period processing unit configured to divide the measurement period into a plurality of sub periods; and
a time series calculating unit configured to calculate a plurality of first prediction data respectively associated with the plurality of sub periods based on first embedding data of the plurality of embedding data.

16. The time series data processing device of claim 15, wherein the time series calculating unit is configured to:
estimate a first slope based on a first sub period of the plurality of sub periods and the first embedding data;
calculate one prediction data of the plurality of first prediction data based on the first slope, the first sub period, and the first embedding data;
estimate a second slope based on a second sub period of the plurality of sub periods and the one prediction data; and
calculate another prediction data of the plurality of first prediction data based on the second slope, the second sub period, and the one prediction data.

17. The time series data processing device of claim 15, wherein the feature irregularity predicting module includes:
a missing value mask processing unit configured to generate masked prediction data based on last prediction data of the plurality of first prediction data and the mask data; and
a missing value replacement applying unit configured to generate replacement data by replacing a missing value of feature data corresponding to the masked prediction data from among the plurality of feature data, based on the masked prediction data.

18. The time series data processing device of claim 17, wherein the time series calculating unit is further configured to:
calculate a plurality of second prediction data respectively associated with the plurality of sub periods based on the replacement data.
